(19) United States
(12) Patent Application Publication
(10) Pub. No.: US 2004/0097522 A1
(43) Pub. Date: May 20, 2004

(54) Triazolopyrimidines

(76) Inventors: Olaf Gebauer, Koln (DE); Hans-Ludwig Elbe, Wuppertal (DE); Marielouise Schneider, Leverkusen (DE); Albrecht Marhold, Leverkusen (DE); Ulrike Wachendorff-Neumann, Neuwied (DE); Astrid Mauler-Machnik, Leichlingen (DE); Karl-Heinz Kuck, Langenfeld (DE); Arnd Voerste, Koln (DE); Yoshinori Kitagawa, Moka-shi (JP); Ulrich Heinemann, Leichlingen (DE); Petra Hilgers, Rosrath (DE); Axel Pleschke, Koln (DE)

Correspondence Address: Bayer CropScience, Patent Department, 100 Bayer Road, Pittsburgh, PA, 15205-9741, US

(21) Appl. No.: 10/450,744

(22) PCT Filed: Dec. 7, 2001
(86) PCT No.: PCT/EP01/14415

(30) Foreign Application Priority Data
Dec. 18, 2000 (DE) 10063115.0

(51) Int. Cl. A61K 31/519; C07D 487/04

(52) U.S. Cl. 514/259.31; 544/263

(57) ABSTRACT

Novel triazolopyrimidines of the formula

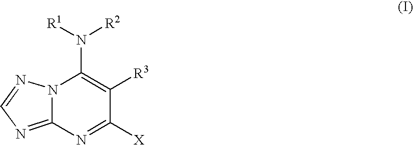

in which

R¹, R², R³ and X have the meanings given in the description,

a process for preparing these novel substances and their use for controlling undesirable microorganisms and animal pests.

Novel amines of the formula

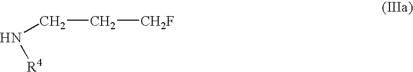

in which

R⁴ has the meanings given in the description,

and processes for their preparation.

The present invention relates to novel triazolopyrimidines, to a process for their preparation and to their use for controlling undesirable microorganisms and animal pests.

It is already known that certain triazolopyrimidines have fungicidal properties (cf. EP-A 0 550 113, WO 94-20 501, EP-A 0 613 900, U.S. Pat. No. 5,612,345, EP-A 0 834 513, WO 98-46 607 and WO 98-46 608). The activity of these substances is good; however, at low application rates, it is sometimes unsatisfactory.

This invention, accordingly, provides novel triazolopyrimidines of the formula

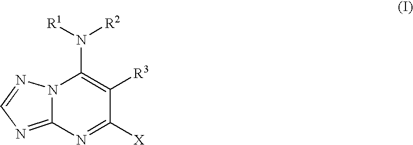

in which

R¹ and R² independently of one another represent alkyl which is mono- or polysubstituted by identical or different substituents from the group consisting of halogen, cyano, nitro, amino, hydroxyl, formyl, carboxyl, carbamoyl, thiocarbamoyl, alkoxy, alkylthio, alkylsulphinyl, alkylsulphonyl, alkenyloxy, alkinyloxy, halogenoalkoxy, halogenoalkylthio, halogenoalkylsulphinyl, halogenoalkylsulphonyl, halogenoalkenyloxy, halogenoalkenylthio, halogenoalkenylsulphinyl, halogenoalkenylsulphonyl, halogenoalkinyloxy, halogenoalkinylthio, halogenoalkinylsulphinyl, halogenoalkinylsulphonyl, alkylamino, dialkylamino, alkylcarbonyl, alkylaminocarbonyl, dialkylaminocarbonyl, alkylaminothiocarbonyl, dialkylaminothiocarbonyl, alkylcarbonyloxy, alkoxycarbonyl, hydroximinoalkyl, alkoximinoalkyl, cycloalkyl, aryl and heterocyclyl,

or

represent optionally substituted alkenyl, optionally substituted alkinyl or optionally substituted cycloalkyl,

R³ represents optionally substituted heterocyclyl or represents substituted aryl, where at least one substituent from the following group of radicals is present

formyl, alkinyl, halogenoalkyl, halogenoalkoxy, halogenoalkylthio, halogenoalkylsulphinyl, halogenoalkylsulphonyl, halogenoalkenyl, halogenoalkinyl, halogenoalkenyloxy, halogenoalkinyloxy, alkylamino, dialkylamino, alkylcarbonyl, alkoxycarbonyl, hydroximinoalkyl or alkoximinoalkyl,

doubly attached alkylene having 3 to 6 carbon atoms which may be mono- or polysubstituted by identical or different substituents from the group consisting of halogen, alkyl having from 1 to 4 carbon atoms and halogenoalkyl having 1 to 4 carbon atoms and 1 to 9 identical or different halogen atoms,

doubly attached oxyalkylene having 2 to 5 carbon atoms which may be mono- or polysubstituted by identical or different substituents from the group consisting of halogen, alkyl having from 1 to 4 carbon atoms and halogenoalkyl having 1 to 4 carbon atoms and 1 to 9 identical or different halogen atoms,

doubly attached dioxyalkylene having 1 to 4 carbon atoms, where the oxygen atoms are not adjacent and where the alkylene chain may be mono- or polysubstituted by identical or different substituents from the group consisting of halogen, alkyl having 1 to 4 carbon atoms and halogenoalkyl having 1 to 4 carbon atoms and 1 to 9 identical or different halogen atoms,

and

X represents halogen.

Furthermore, it has been found that triazolopyrimidines of the formula (I) can be prepared by reacting dihalogeno-triazolopyrimidines of the formula

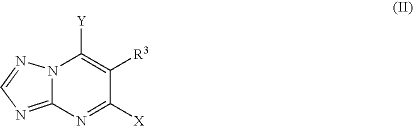

in which

R³ and X have the meanings given above and

Y represents halogen

with amines of the formula

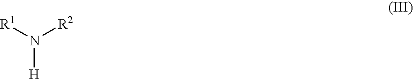

in which

R¹ and R² have the meanings given above,

if appropriate in the presence of a diluent and if appropriate in the presence of an acid acceptor.

Finally, it has been found that the novel triazolopyrimidines of the formula (I) are highly suitable for controlling undesirable microorganisms and animal pests. In particular, they have strong fungicidal and insecticidal activity.

Surprisingly, the triazolopyrimidines of the formula (I) according to the invention have considerably better fungicidal and insecticidal activity than the constitutionally more similar prior-art substances of the same direction of action.

The formula (I) provides a general definition of the triazolopyrimidines according to the invention. Preference is given to those substances of the formula (I) in which

R¹ and R² independently of one another represent alkyl having 1 to 6 carbon atoms, where the alkyl radicals may be mono- to trisubstituted by identical or different substituents from the group consisting of fluorine, chlorine, bromine, cyano, nitro, amino, hydroxyl, formyl, carboxyl, carbamoyl, thiocarbamoyl, alkoxy having 1 to 6 carbon atoms, alkylsulphinyl having 1 to 6 carbon atoms, alkylsulphonyl having 1 to 6 carbon atoms, alkenyloxy having 2 to 6 carbon atoms, alkinyloxy having 2 to 6 carbon atoms, halogenoalkoxy having 1 to 6 carbon atoms and 1 to 9 halogen atoms, halogenoalkylthio having 1 to 6 carbon atoms and 1 to 9 halogen atoms, halogenoalkylsulphinyl having 1 to 6 carbon atoms and 1 to 9 halogen atoms, halogenoalkylsulphonyl having 1 to 6 carbon atoms and 1 to 9 halogen atoms, halogenoalkenyloxy having 2 to 6 carbon atoms and 1 to 9 halogen atoms, halogenoalkenylthio having 2 to 6 carbon atoms and 1 to 9 halogen atoms, halogenoalkenylsulphinyl having 2 to 6 carbon atoms and 1 to 9 halogen atoms, halogenoalkenylsulphonyl having 2 to 6 carbon atoms and 1 to 9 halogen atoms, halogenoalkinyloxy having 2 to 6 carbon atoms and 1 to 9 halogen atoms, halogenoalkinylthio having 2 to 6 carbon atoms and 1 to 9 halogen atoms, halogenoalkinylsulphinyl having 2 to 6 carbon atoms and 1 to 9 halogen atoms, halogenoalkinylsulphonyl having 2 to 6 carbon atoms and 1 to 9 halogen atoms, alkylamino having 1 to 6 carbon atoms, dialkylamino having 1 to 6 carbon atoms in each alkyl moiety, alkylcarbonyl having 1 to 6 carbon atoms in the alkyl moiety, alkylaminocarbonyl having 1 to 6 carbon atoms in the alkyl moiety, dialkylaminocarbonyl having 1 to 6 carbon atoms in each alkyl moiety, alkylaminothiocarbonyl having 1 to 6 carbon atoms in the alkyl moiety, dialkylaminothiocarbonyl having 1 to 6 carbon atoms in each alkyl moiety, alkylcarbonyloxy having 1 to 6 carbon atoms in the alkyl moiety, alkoxycarbonyl having 1 to 6 carbon atoms in the alkoxy moiety, hydroximinoalkyl having 1 to 6 carbon atoms, alkoximinoalkyl having 1 to 6 carbon atoms in the alkoxy moiety and 1 to 6 carbon atoms in the alkyl moiety, cycloalkyl having 3 to 6 carbon atoms, aryl having 6 to 10 carbon atoms and/or heterocyclyl having 3 to 8 ring members and 1 to 3 heteroatoms, such as nitrogen, oxygen and/or sulphur,

or

represent alkenyl having 2 to 6 carbon atoms, alkinyl having 2 to 6 carbon atoms or cycloalkyl having 3 to 6 carbon atoms, where the three abovementioned radicals may be mono- to trisubstituted by identical or different substituents from the group consisting of fluorine, chlorine, bromine, cyano, nitro, amino, hydroxyl, formyl, carboxyl, carbamoyl, thiocarbamoyl, alkoxy having 1 to 6 carbon atoms, alkylthio having 1 to 6 carbon atoms, alkylsulphinyl having 1 to 6 carbon atoms, alkylsulphonyl having 1 to 6 carbon atoms, alkenyloxy having 2 to 6 carbon atoms, alkinyloxy having 2 to 6 carbon atoms, halogenoalkoxy having 1 to 6 carbon atoms and 1 to 9 halogen atoms, halogenoalkylthio having 1 to 6 carbon atoms and 1 to 9 halogen atoms, halogenoalkylsulphinyl having 1 to 6 carbon atoms and 1 to 9 halogen atoms, halogenoalkylsulphonyl having 1 to 6 carbon atoms and 1 to 9 halogen atoms, halogenoalkenyloxy having 2 to 6 carbon atoms and 1 to 9 halogen atoms, halogenoalkenylthio having 2 to 6 carbon atoms and 1 to 9 halogen atoms, halogenoalkenylsulphinyl having 2 to 6 carbon atoms and 1 to 9 halogen atoms, halogenoalkenylsulphonyl having 2 to 6 carbon atoms and 1 to 9 halogen atoms, halogenoalkinyloxy having 2 to 6 carbon atoms and 1 to 9 halogen atoms, halogenoalkinylthio having 2 to 6 carbon atoms and 1 to 9 halogen atoms, halogenoalkinylsulphinyl having 2 to 6 carbon atoms and 1 to 9 halogen atoms, halogenoalkinylsulphonyl having 2 to 6 carbon atoms and 1 to 9 halogen atoms, alkylamino having 1 to 6 carbon atoms, dialkylamino having 1 to 6 carbon atoms in each alkyl moiety, alkyl carbonyl having 1 to 6 carbon atoms in the alkyl moiety, alkylcarbonyloxy having 1 to 6 carbon atoms in the alkyl moiety, alkoxycarbonyl having 1 to 6 carbon atoms in the alkoxy moiety, alkylaminocarbonyl having 1 to 6 carbon atoms in the alkyl moiety, dialkylaminocarbonyl having 1 to 6 carbon atoms in each alkyl moiety, alkylaminothiocarbonyl having 1 to 6 carbon atoms in the alkyl moiety, dialkylaminothiocarbonyl having 1 to 6 carbon atoms in each alkyl moiety, hydroximinoalkyl having 1 to 6 carbon atoms, alkoximinoalkyl having 1 to 6 carbon atoms in the alkoxy moiety and 1 to 6 carbon atoms in the alkyl moiety, cycloalkyl having 3 to 6 carbon atoms, aryl having 6 to 10 carbon atoms and/or heterocyclyl having 3 to 8 ring members and 1 to 3 heteroatoms, such as nitrogen, oxygen and/or sulphur.

R³ represents five- or six-membered heterocyclyl having 1 to 4 heteroatoms, such as nitrogen, oxygen and/or sulphur, it being possible for these radicals to be mono- to trisubstituted by identical or different substituents from the group consisting of

halogen, cyano, nitro, formyl;

in each case straight-chain or branched alkyl, alkoxy, alkylthio, alkylsulphinyl or alkylsulphonyl having in each case 1 to 6 carbon atoms;

in each case straight-chain or branched alkenyl, alkinyl, alkenyloxy or alkinyloxy having in each case 2 to 6 carbon atoms;

in each case straight-chain or branched halogenoalkyl, halogenoalkoxy, halogenoalkylthio, halogenoalkylsulphinyl or halogenoalkylsulphonyl having in each case 1 to 6 carbon atoms and 1 to 13 identical or different halogen atoms;

in each case straight-chain or branched halogenoalkenyl, halogenoalkinyl, halogenoalkenyloxy or halogenoalkinyloxy having in each case 2 to 6 carbon atoms and 1 to 9 identical or different halogen atoms;

in each case straight-chain or branched alkylamino, dialkylamino, alkylcarbonyl, alkylcarbonyloxy, alkoxycarbonyl, hydroximinoalkyl or alkoximinoalkyl having in each case 1 to 6 carbon atoms in the individual alkyl moieties;

and/or

cycloalkyl having 3 to 6 carbon atoms,

or

R³ represents aryl having 6 to 10 carbon atoms which is mono- to pentasubstituted by identical or different substituents from the group consisting of

halogen, cyano, nitro, formyl;

in each case straight-chain or branched alkyl, alkoxy, alkylthio, alkylsulphinyl or alkylsulphonyl having in each case 1 to 6 carbon atoms;

in each case straight-chain or branched alkenyl, alkinyl, alkenyloxy or alkinyloxy having in each case 2 to 6 carbon atoms;

in each case straight-chain or branched halogenoalkenyl, halogenoalkinyl, halogenoalkenyloxy or halogenoalkinyloxy having in each case 1 to 6 carbon atoms and 1 to 13 identical or different halogen atoms;

in each case straight-chain or branched halogenoalkyl, halogenoalkinyl, halogenoalkenyloxy or halogenoalkinyloxy having in each case 2 to 6 carbon atoms and 1 to 13 identical or different halogen atoms;

in each case straight-chain or branched alkylamino, dialkylamino, alkylcarbonyl, alkylcarbonyloxy, alkoxycarbonyl, hydroximinoalkyl or alkoximinoalkyl having in each case 1 to 6 carbon atoms in the individual alkyl moieties;

cycloalkyl having 3 to 6 carbon atoms;

doubly attached alkylene having 3 or 4 carbon atoms which may be mono- to tetrasubstituted by identical or different substituents from the group consisting of fluorine, chlorine, bromine, methyl, ethyl and/or halogenoalkyl having 1 or 2 carbon atoms and 1 to 3 fluorine, chlorine and/or bromine atoms,

doubly attached oxyalkylene having 2 or 3 carbon atoms which may be mono- to tetrasubstituted by identical or different substituents from the group consisting of fluorine, chlorine, bromine, methyl, ethyl and halogenoalkyl having 1 or 2 carbon atoms and 1 to 3 fluorine, chlorine and/or bromine atoms,

and/or

doubly attached dioxyalkylene having 1 or 2 carbon atoms, where the oxygen atoms are not adjacent and where the alkylene chain may be mono- to tetrasubstituted by identical or different substituents from the group consisting of fluorine, chlorine, bromine, methyl, ethyl and halogenoalkyl having 1 or 2 carbon atoms and 1 to 3 fluorine, chlorine and/or bromine atoms,

where at least one substituent from the following group of radicals is present:

formyl,

in each case straight-chain or branched halogenoalkyl, halogenoalkoxy, halogenoalkylthio, halogenoalkylsulphinyl or halogenoalkylsulphonyl having in each case 1 to 6 carbon atoms and 1 to 13 identical or different halogen atoms;

in each case straight-chain or branched halogenoalkenyl, halogenoalkinyl, halogenoalkenyloxy or halogenoalkinyloxy having in each case 2 to 6 carbon atoms and 1 to 13 identical or different halogen atoms;

in each case straight-chain or branched alkylamino, dialkylamino, alkylcarbonyl, alkoxycarbonyl, hydroximinoalkyl or alkoximinoalkyl having in each case 1 to 6 carbon atoms in the individual alkyl moieties;

doubly attached alkylene having 3 or 4 carbon atoms which may be mono- to tetrasubstituted by identical or different substituents from the group consisting of fluorine, chlorine, bromine, methyl, ethyl and/or halogenoalkyl having 1 or 2 carbon atoms and 1 to 3 fluorine, chlorine and/or bromine atoms,

doubly attached oxyalkylene having 2 or 3 carbon atoms which may be mono- to tetrasubstituted by identical or different substituents from the group consisting of fluorine, chlorine, bromine, methyl, ethyl and halogenoalkyl having 1 or 2 carbon atoms and 1 to 3 fluorine, chlorine and/or bromine atoms,

doubly attached dioxyalkylene having 1 or 2 carbon atoms, where the oxygen atoms are not adjacent and where the alkylene chain may be mono- to tetrasubstituted by identical or different substituents from the group consisting of fluorine, chlorine, bromine, methyl, ethyl and halogenoalkyl having 1 or 2 carbon atoms and 1 to 3 fluorine, chlorine and/or bromine atoms,

and

X represents fluorine, chlorine or bromine.

Particular preference is given to those substances of the formula (I) in which

R¹ and R² independently of one another represent methyl, ethyl, n-propyl, isopropyl, n-, i-, sec.- or tert.-butyl, pentyl or hexyl, where these radicals are mono- to trisubstituted by identical or different substituents from the group consisting of fluorine, chlorine, bromine, cyano, nitro, amino, hydroxyl, formyl, carboxyl, carbamoyl, thiocarbamoyl, methoxy, ethoxy, n- or i-propoxy, methylthio, ethylthio, n- or i-propylthio, methylsulphinyl, ethylsulphinyl, methylsulphonyl or ethylsulphonyl, allyloxy, propargyloxy, difluoromethoxy, trifluoromethoxy, difluorochloromethoxy, trifluoroethoxy, difluoromethylthio, difluorochloromethylthio, trifluoromethylthio, trifluoromethylsulphinyl or trifluoromethylsulphonyl, trichloroethinyloxy, trifluoroethinyloxy, chloroallyloxy, iodopropargyloxy, trichloroethinylthio, trifluoroethinylthio, chloroallylthio, iodopropargylthio, trichloroethinylsulphinyl, trifluoroethinylsulphinyl, chloroallylsulphinyl, iodopropargylsulphinyl, trichloroethinylsulphonyl, trifluoroethinylsulphonyl, chloroallylsulphonyl, iodopropargylsulphonyl, methylamino, ethylamino, n- or i-propylamino, dimethylamino, diethylamino, acetyl, propionyl, methylaminocarbonyl, ethylaminocarbonyl, n- or i-propylaminocarbonyl, dimethylaminocarbonyl, diethylaminocarbonyl, methylaminothiocarbonyl, ethylaminothiocarbonyl, n- and i-propylaminothiocarbonyl, dimethylaminothiocarbonyl, diethylaminothiocarbonyl, acetyloxy, methoxycarbonyl, ethoxycarbonyl, hydroximinomethyl, hydroximinoethyl, methoximinomethyl, ethoximinomethyl, methoximinoethyl or ethoximinoethyl, cyclopropyl, cyclobutyl, cyclopentyl or cyclohexyl, phenyl, pyridyl, furyl, thienyl, dioxolanyl, oxazolidinyl, dithiolanyl, methyldioxolanyl, methyloxazolidinyl and methyldithiolanyl

or

R¹ and R² independently of one another represent ethenyl, propenyl, butenyl, pentenyl, hexenyl, ethinyl, propinyl, butinyl, hexinyl, cyclopropyl, cyclobutyl, cyclopentyl, where these radicals may be mono- to trisubstituted by identical or different substituents from the group consisting of fluorine, chlorine, bromine, cyano, nitro, amino, hydroxyl, formyl, carboxyl, carbamoyl, thiocarbamoyl, methoxy, ethoxy, n- or i-propoxy, methylthio, ethylthio, n- or i-propylthio, methylsulphinyl, ethylsulphinyl, methylsulphonyl, ethylsulphonyl, allyloxy, propargyloxy, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethinyloxy, trifluoroethinyloxy, chloroallyloxy, iodopropargyloxy, trichloroethinylthio, trifluoroethinylthio, chloroallylthio, iodopropargylthio, trichloroethinylsulphinyl, trifluoroethinylsulphinyl, chloroallylsulphinyl, iodopropargylsulphinyl, trichloroethinylsulphonyl, trifluoroethinylsulphonyl, chloroallylsulphonyl, iodopropargylsulphonyl, methylamino, ethylamino, n- or i-propylamino, dimethylamino, diethylamino, acetyl, propionyl, methylaminocarbonyl, ethylaminocarbonyl, n- or i-propylaminocarbonyl, dimethylaminocarbonyl, diethylaminocarbonyl, methylaminothiocarbonyl, ethylaminothiocarbonyl, nor i-propylaminothiocarbonyl, dimethylaminothiocarbonyl, diethylaminothiocarbonyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl or cyclohexyl, phenyl, pyridyl, furyl thienyl, dioxolanyl, oxazolidinyl, dithiolanyl, methyldioxolanyl, methyloxazolidinyl and methyldithiolanyl,

R³ represents furyl, thienyl, pyridyl or pyrimidyl, where these radicals may be mono- to trisubstituted by identical or different constituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, ethylsulphinyl, methylsulphonyl, ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethinyloxy, trifluoroethinyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl or ethoximinoethyl, cyclopropyl, cyclobutyl, cyclopentyl and cyclohexyl,

or

R³ represents phenyl which is mono- to pentasubstituted by identical or different substituents from the group consisting of fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, ethylsulphinyl, methylsulphonyl or ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethinyloxy, trifluoroethinyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl and ethoximinoethyl,

in each case doubly attached propane-1,3-diyl, methylenedioxy or ethylenedioxy, where these radicals may be mono- to tetrasubstituted by identical or different substituents from the group consisting of fluorine, chlorine, methyl, ethyl, n-propyl, i-propyl and trifluoromethyl, cyclopropyl, cyclobutyl, cyclopentyl or cyclohexyl,

where at least one substituent from the following group of radicals is present:

formyl,

trifluoromethyl, trifluoroethyl, difluoromethoxy, trifluoromethoxy, difluoromethoxy, trifluoroethoxy, difluoromethylthio, difluorochloromethylthio, trifluoromethylthio, methylamino, ethylamino, n- or i-propylamino, dimethylamino, diethylamino, methoxycarbonyl, ethoxycarbonyl, hydroximinomethyl, hydroximinoethyl, methoximinomethyl, ethoximinomethyl, methoximinoethyl or ethoximinoethyl,

in each case doubly attached propane-1,3-diyl, methylenedioxy or ethylenedioxy, where these radicals may be mono- to tetrasubstituted by identical or different substituents from the group consisting of fluorine, chlorine, methyl, ethyl, n-propyl, i-propyl and trifluoromethyl,

and

X represents fluorine or chlorine.

The radical definitions mentioned above can be combined with one another as desired. Moreover, individual meanings may not apply.

A very particularly preferred group of compounds according to the invention are those triazolopyrimidines of the formula (I), in which

R¹ represents methyl, ethyl, n-propyl, isopropyl, n-, i-, sec- or tert-butyl, it being possible for these radicals to be mono- to trisubstituted by identical or different substituents from the group consisting of fluorine, chlorine, bromine, methoxy, ethoxy, acetyl, dimethylaminocarbonyl, methoxycarbonyl, ethoxycarbonyl and phenyl,

or

R¹ represents propenyl, butenyl, pentenyl or hexenyl, it being possible for these radicals to be mono- to trisubstituted by identical or different substituents from the group consisting of fluorine, chlorine and bromine,

R² represents methyl, ethyl, n-propyl, isopropyl, n-, i-, sec- or tert-butyl, it being possible for these radicals to be mono- to trisubstituted by identical or different substituents from the group consisting of fluorine, chlorine and bromine,

R³ represents phenyl which is mono- to tetrasubstituted by identical or different constituents from the group consisting of fluorine, chlorine, trifluoromethyl and trifluoromethylthio,

where at least one substituent is a trichloromethyl or trichloromethylthio group, or

R³ represents the radical of the formula

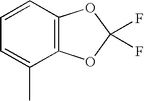

and

X represents fluorine or chlorine.

Using 5,7-dichloro-6-(2,6-dichloro-4-trifluoromethyl-phenyl)-[1,2,4]-triazolo-[1,5-a]pyrimidine and 2,2,2-trifluoroethyl-3-fluoro-propyl-amine as starting materials, the course of the process according to the invention can be illustrated by the formula scheme below.

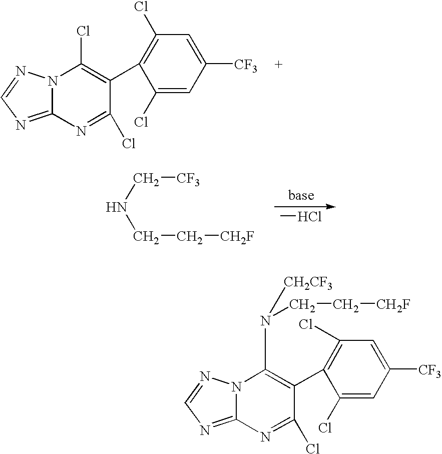

The formula (II) provides a general definition of the dihalogeno-triazolopyrimidines required as starting materials for carrying out the process according to the invention. In this formula, R³ and X preferably have those meanings which have already been mentioned in connection with the description of the substances of the formula (I) according to the invention as being preferred for these radicals. Y preferably represents fluorine, chlorine or bromine, particularly preferably fluorine or chlorine.

Examples of dihalogeno-triazolopyrimidines of the formula (II) which may be mentioned are the substances of the formulae below.

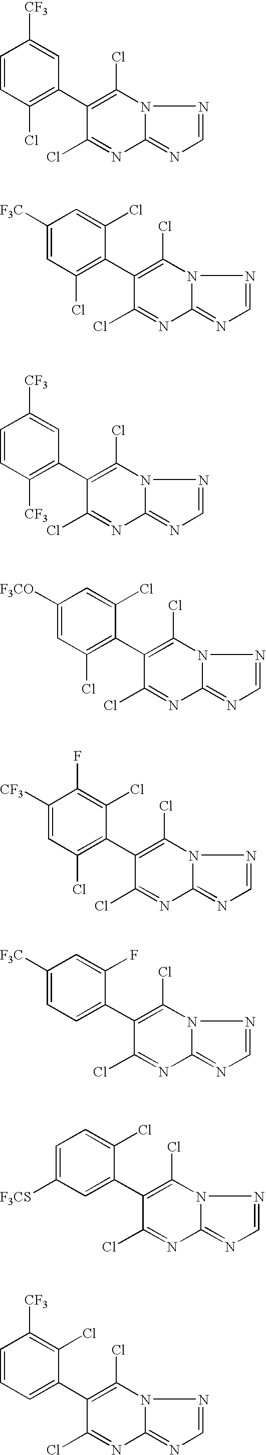

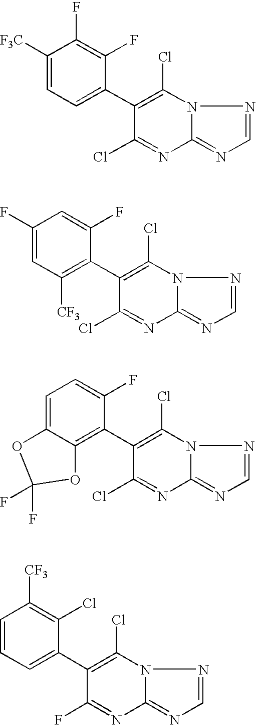

The dihalogeno-triazolopyrimidines of the formula (II) are known or can be prepared by known methods (cf. U.S. Pat. No. 5,808,066, U.S. Pat. No. 5,612,345 and WO 94-20 501).

The formula (III) provides a general definition of the amines furthermore required as starting materials for carrying out the process according to the invention. In this formula, R¹ and R² preferably have those meanings which have already been mentioned in connection with the description of the substances of the formula (I) according to the invention as being preferred for these radicals.

Amines of the formula

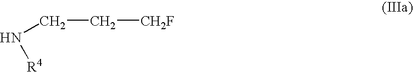

in which

R⁴ represents the radicals of the formulae

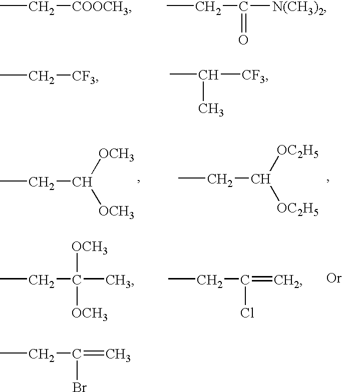

are novel.

Amines of the formula (IIIa) can be prepared by reacting either

a) 1-fluoro-3-bromo-propane of the formula

FCH₂—CH₂—CH₂—Br (IV)

with amines of the formula

H₂N—R⁴ (V)

in which

R⁴ has the meanings given above,

or with acid addition salts of amines of the formula (V),

in the presence of an inert organic diluent, such as, for example, acetonitrile, and in the presence of an acid binder, such as, for example, potassium carbonate, at temperatures between 10° C. and 80° C.,

or

b) 3-fluoro-propyl-amine of the formula

FCH₂—CH₂—CH₂—NH₂ (VI),

if appropriate in the form of an acid addition salt,

with halogen compounds of the formula

R⁴—Hal (VII)

in which

R⁴ has the meanings given above and

Hal represents chlorine or bromine

in the presence of an inert diluent, such as, for example, acetonitrile, and in the presence of an acid binder, such as, for example, potassium carbonate, at temperatures between 10° C. and 80° C.

The 1-fluoro-3-bromo-propane of the formula (IV) required as starting material for carrying out the process (a) has hitherto not been disclosed. It can be prepared by initially reacting 3-chloro-1-proponal with potassium fluroide in the presence of a diluent, such as, for example, triethylene glycol, at temperatures between 50° C. and 110° C., and then, in a second step, reacting the resulting 3-fluoro-1-propanol with thionyl bromide in the presence of a diluent, such as, for example, dimethylformamide, at temperatures between 0° C. and 30° C.

The amines of the formula (V) required as reaction components for carrying out the process (a) are used either as such or in the form of acid addition salts. Preference is given here to ammonium salts formed by addition of hydrogen chloride or tosyl chloride to amines of the formula (V).

The amines of the formula (V) and their acid addition salts are known or can be prepared by known methods.

The 3-fluoropropyl-amine of the formula (VI) required as starting material for carrying out the process (b) and its acid addition salts can be prepared by initially reacting 3-fluoro-1-propanol with tosyl chloride in the presence of an acid binder, such as, for example, pyridine, at temperatures between −10° C. and +10° C., and then, in a second step, treating the resulting 3-fluoropropyl-tosylate in the presence of a diluent, such as, for example, ethanol, at temperatures between 10° C. and 30° C., with ammonia. The amine can be released from the resulting 3-fluoropropyl-ammonium tosylate by treatment with aqueous alkali metal hydroxide. The amine can be converted by reaction with acids into the corresponding acid addition salts.

Preference is given to those ammonium salts which are formed by addition of hydrogen chloride or tosyl chloride to the 3-fluoropropyl-amine of the formula (VI).

The halogen compounds of the formula (VII) required as reaction components for carrying out the process (b) are known or can be prepared by known methods.

Analogously to the amines of the formula (IIIa), it is also possible to prepare the other amines of the formula (III).

Suitable diluents for carrying out the process according to the invention are all customary inert organic solvents. Preference is given to using aliphatic, alicyclic or aromatic hydrocarbons, such as petroleum ether, hexane, heptane, cyclohexane, methylcyclohexane, benzene, toluene, xylene or decaline; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane.

Suitable acid acceptors for carrying out the process according to the invention are also acid binders which are customary for such reactions.

Preference is given to using tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process according to the invention, the reaction temperatures can be varied within a relatively wide range. In general, the process is carried out at temperatures between 0° C. and 150° C., preferably at temperatures between 0° C. and 80° C.

When carrying out the process according to the invention, in general from 1 to 15 mol, preferably from 2 to 8 mol, of amine of the formula (III) are employed per mole of dihalogeno-triazolopyrimidine of the formula (II). Work-up is carried out by customary methods.

The process according to the invention is generally carried out under atmospheric pressure. However, it is also possible to operate under elevated or reduced pressure, generally between 0.1 bar and 10 bar.

The substances according to the invention have a powerful microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidial form: Drechslera, syn: Helminthosporium);

Cochliobolus species, such as, for example, Cochliobolus sativus (conidial form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae;

Pseudocercosporella species, such as, for example, Pseudocercosporella herpotrichoides.

The good plant tolerance of the active compounds according to the invention at the concentrations required for controlling plant diseases allows treatment of above-ground parts of plants, of propagation stock and seeds, and of the soil.

The active compounds according to the invention can be employed particularly successfully for controlling cereal diseases, such as, for example, against Erysiphe or Puccinia species, diseases in viticulture and fruit and vegetable production such as, for example, against Venturia, Uncinula and Podosphaera species, or rice diseases, such as, for example, against Pyricularia species.

The active compounds according to the invention are also suitable for increasing the yield. Moreover, they exhibit low toxicity and are well tolerated by plants.

In the protection of materials, the substances according to the invention can be employed for protecting industrial materials against infection with, and destruction by, undesirable microorganisms.

Industrial materials in the present context are understood as meaning non-living materials which have been prepared for use in industry. For example, industrial materials which are intended to be protected by active compounds according to the invention from microbial change or destruction can be glues, sizes, paper and board, textiles, leather, wood, paints and synthetic articles, cooling lubricants and other materials which can be infected with, or destroyed by, microorganisms. Parts of production plants, for example cooling-water circuits, which may be impaired by the multiplication of microorganisms may also be mentioned within the scope of the materials to be protected. Industrial materials which may be mentioned within the scope of the present invention are preferably glues, sizes, paper and board, leather, wood, paints, cooling lubricants and heat-transfer liquids, especially preferably wood.

Microorganisms of the following genera may be mentioned by way of example:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

The active compounds according to the invention are also suitable for controlling animal pests, in particular insects, arachnids and nematodes found in agriculture, in forests, in horticulture, in the protection of stored products and materials and in the hygiene sector or in the field of veterinary medicine, while being well tolerated by plants and exhibiting an advantageous toxicity to warm-blooded species. The substances are effective against normally sensitive and resistant species and against pests in all or individual developmental stages. The abovementioned animal pests include:

From the order of the Isopoda, for example, Oniscus asellus, Armadillidium vulgare, Porcellio scaber.

From the order of the Diplopoda, for example, Blaniulus guttulatus.

From the order of the Chilopoda, for example, Geophilus carpophagus, Scutigera spec.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blatta orientalis, Periplaneta americana, Leucophaea maderae, Blattella germanica, Acheta domesticus, Gryllotalpa spp., Locusta migratoria migratorioides, Melanoplus differentialis, Schistocerca gregaria.

From the order of the Dermaptera, for example, Forficula auricularia.

From the order of the Isoptera, for example, Reticulitermes spp.

From the order of the Anoplura, for example, Pediculus humanus corporis, Haematopinus spp., Linognathus spp.

From the order of the Mallophaga, for example, Trichodectes spp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Spodoptera exigua, Mamestra brassicae, Panolis flammea, Spodoptera litura, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopis, Ceratophyllus spp.

From the order of the Arachnida, for example, Scorpio maurus, Latrodectus mactans.

From the order of the Acarina, for example, Acarus siro, Argas spp., Omithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp.

The substances which can be used in accordance with the invention can be employed particularly successfully for controlling plant-damaging mites, such as against the greenhouse red spider mite (Tetranychus urticae), or for controlling plant-injurious insects, such as against the larvae of the mustard beetle (Phaedon cochleariae), and of the green peach aphid (Mycus persicae).

In addition, the compounds of the formula (I) according to the invention also exhibit a very good antimycotic activity. They have a very broad antimycotic spectrum of action, in particular against Dermatophytes and yeasts, moulds and diphasic fungi (for example against Candida species such as Candida albicans, Candida glabrata), Epidermophyton species such as Epidermophyton floccosum, Aspergillus species such as Aspergillus niger and Aspergillus fumigatus, Trichophyton species such as Trichophyton mentagrophytes, Microsporon species, such as Microsporon canis and audouinii. This enumeration of fungi constitutes in no way a limitation of the mycotic spectrum which can be controlled, but has merely illustrative character.

Depending on their particular physical and/or chemical properties, the active compounds can be converted into the customary formulations, such as solutions, emulsions, suspensions, powders, foams, pastes, granules, aerosols, macroencapsulations in polymeric substances and in coating compositions for seed, and ULV cold and hot formulations.

These formulations are produced in a known manner, for example by mixing the active compounds with extenders, that is liquid solvents, liquefied gases under pressure and/or solid carriers, optionally with the use of surfactants, that is, emulsifiers and/or dispersants and/or foam formers. In the case of the use of water as extender, organic solvents can, for example, also be used as cosolvents. The following are mainly suitable as liquid solvents: aromatics such as xylene, toluene or alkylnaphthalenes, chlorinated aromatics or chlorinated aliphatic hydrocarbons, such as chlorobenzenes, chloroethylenes or methylene chloride, aliphatic hydrocarbons such as cyclohexane or paraffins, for example mineral oil fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water. Liquefied gaseous extenders or carriers are to be understood as meaning liquids which are gaseous at ambient temperature and under atmospheric pressure, for example aerosol propellants such as halogenohydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example non-ionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example ligninsulphite waste liquors and methylcellulose.

Adhesives such as carboxymethylcellulose, and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compounds, preferably between 0.5 and 90%.

The active compounds according to the invention, as such or in their formulations, can also be used in a mixture with known fungicides, bactericides, acaricides, nematicides or insecticides, for example to widen the spectrum of action or to prevent the development of resistance. In many cases, synergistic effects are obtained, that is the activity of the mixture is greater than the activity of the individual components.

Examples of suitable components in mixtures are the following:

Fungicides:

aldimorph, ampropylfos, ampropylfos-potassium, andoprim, anilazin, azaconazole, azoxystrobin,

benalaxyl, benodanil, benomyl, benzamacril, benzamacryl-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamid,

debacarb, dichlorophen, diclobutrazole, diclofluanid, diclomezin, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

ediphenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromid, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fenhexamide,

guazatine,

hexachlorobenzene, hexaconazole, hymexazole,

imazalil, imibenconazole, iminoctadin, iminoctadin albesilate, iminoctadin triacetate, iodocarb, ipconazole, iprobenfos (IBP), iprodione, irumamycin, isoprothiolan, isovaledione, iprovalicarb,

kasugamycin, kresoxim-methyl, copper preparations such as: copper hydroxide, copper naphthenate, copper oxychloride, copper sulfate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidon, propamocarb, propanosine-sodium, propiconazole, propineb, pyrazophos, pyrifenox, pyrimethanil, pyroquilon, pyroxyfur,

quinconazole, quintozene (PCNB), quinoxyfen,

sulphur and sulphur preparations, spiroxamine,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforin, triticonazole, trifloxystrobin,

uniconazole,

validamycin A, vinclozolin, viniconazole,

zarilamid, zineb, ziram and

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H- 1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinol,

2′,6′-dibromo-2-methyl4′-trifluoromethoxy4-trifluoro-methyl-1,3-thiazole-5carboxanilide,

2,6-dichloro-5-(methylthio)-4-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol(OPP),

3,4-dichloro-1-[4-(difluoromethoxy)-phenyl]-1H-pyrrole-2,5-dione,

3,5-dichloro-N-[cyano[(1-methyl-2-propynyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-hydroxyquinoline sulphate,

N-2-[(phenylamino)-carbonyl]-9H-xanthene-9-carbohydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine hydrochloride,

ethyl [(4-chlorophenyl)-azo]-cyanoacetate,

potassium hydrogen carbonate,

sodium methanetetrathiolate,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)-acetamide,

N-(6-methoxy)-3-pyridinyl)-cyclopropanecarboxamide,

N-[2,2,2-trichloro-1-[(chloroacetyl)-amino]-ethyl]-benzamide,

N-[3-chloro-4,5-bis-(2-propinyloxy)-phenyl]-N′-methoxy-methaneimidamide,

sodium N-formyl-N-hydroxy-DL-alaninate,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran]-3′-one,

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/acaricides/nematicides:

abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, diflubenzuron, dimethoate, dimethylvinphos, diofenolan, disulfoton, docusate sodium, dofenapyn,

eflusilanate, emamectin, empenthrin, endosulfan, Entomophthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flutenzine, fluvalinate, fonophos, fosmethilan, fosthiazate, fubfenprox, furathiocarb,

granulosis viruses

halofenozide, HCH, heptenophos, hexaflumuron, hexythiazox, hydroprene,

imidacloprid, isazofos, isofenphos, isoxathion, ivermectin,

nuclear polyhedrosis viruses

lambda-cyhalothrin, lufenuron

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron

omethoate, oxamyl, oxydemethon M

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propoxur, prothiofos, prothoate, pymetrozine, pyraclofos, pyresmethrin, pyrethrum, pyridaben, pyridathion, pyrimidifen, pyriproxyfen,

quinalphos,

ribavirin

salithion, sebufos, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii

YI 5302

zeta-cypermethrin, zolaprofos

(1R-cis)-[5-(phenylmethyl)-3-furanyl]-methyl-3-[(dihydro-2-oxo-3(2H)-furanylidene)-methyl]-2,2-dimethylcyclopropanecarboxylate

(3-phenoxyphenyl)-methyl 2,2,3,3-tetramethylcyclopropanecarboxylate

1-[(2-chloro-5-thiazolyl)methyl]tetrahydro-3,5-dimethyl-N-nitro-1,3,5-triazine-2(1H)-imine

2-(2-chloro-6-fluorophenyl)-4-[4-(1,1-dimethylethyl)phenyl]-4,5-dihydro-oxazole

2-(acetyloxy)-3-dodecyl-1,4-naphthalenedione

2-chloro-N-[[[4-(1-phenylethoxy)-phenyl]-amino]-carbonyl]-benzamide

2-chloro-N-[[[4-(2,2-dichloro-1,1-difluoroethoxy)-phenyl]-amino]-carbonyl]-benzamide

3-methylphenyl propylcarbamate

4-[4-(4-ethoxyphenyl)-4-methylpentyl]-1-fluoro-2-phenoxy-benzene

4-chloro-2-(1,1-dimethylethyl)-5-[[2-(2,6-dimethyl-4-phenoxyphenoxy)ethyl]thio]-3(2H)-pyridazinone

4-chloro-2-(2-chloro-2-methylpropyl)-5-[(6-iodo-3-pyridinyl)methoxy]-3(2H)-pyridazinone

4-chloro-5-[(6-chlor-3-pyridinyl)methoxy]-2-(3,4-dichlorophenyl)-3(2H)-pyridazinone

Bacillus thuringiensis strain EG-2348

N-[2-benzoyl-1-(1,1-dimethylethyl)]-benzohydrazide

2,2-dimethyl-3-(2,4-dichlorophenyl)-2-oxo-1-oxaspiro[4.5]dec-3-en-4-yl butanoate

N-[3-[(6-chloro-3-pyridinyl)methyl]-2-thiazolidinylidene]-cyanamide

dihydro-2-(nitromethylene)-2H-1,3-thiazine-3(4H)-carboxaldehyde

ethyl [2-[[1,6-dihydro-6-oxo-1-(phenylmethyl)-4-pyridazinyl]oxy]ethyl]-carbamate

N-(3,4,4-trifluoro-1-oxo-3-butenyl)-glycine

N-(4-chlorophenyl)-3-[4-(difluoromethoxy)phenyl]-4,5-dihydro-4-phenyl-1H-pyrazole-1-carboxamide

N-[(2-chloro-5-thiazolyl)methyl]-N′-methyl-N″-nitro-guanidine

N-methyl-N′-(1-methyl-2-propenyl)-1,2-hydrazinedicarbothioamide

N-methyl-N′-2-propenyl-1,2-hydrazinedicarbothioamide

O,O-diethyl [2-(dipropylamino)-2-oxoethyl]-ethylphosphoramidothioate

A mixture with other known active compounds such as herbicides, or with fertilizers and growth regulators, is also possible.

All plants and plant parts can be treated with the substances according to the invention. Plants in this context are taken to mean all plants and plant populations, such as desired and undesired wild plants or crop plants (including naturally occurring crop plants). Crop plants can be plants which can be obtained by conventional breeding and optimization methods, or by biotechnological and genetic engineering methods, or by combinations of these methods, including the transgenic plants and including the plant varieties which are capable, or incapable, of being protected by Plant Breeders' Rights. Plant parts are to be taken to mean all above-ground and below-ground parts and organs of the plants, such as shoot, leaf, flower and root; examples mentioned being leaves, needles, stalks, stems, flowers, fruit bodies, fruits and seeds, and also roots, tubers and rhizomes. The plant parts also include harvested material and vegetative and generative propagation material, for example cuttings, tubers, rhizomes, offshoots and seeds.

The active compounds can be used as such or in the form of their formulations or the use forms prepared therefrom, such as ready-to-use solutions, suspensions, wettable powders, pastes, soluble powders, dusts and granules. They are used in the customary manner, for example by pouring, spraying, atomizing, broadcasting, dusting, foaming, brushing-on and the like. It is furthermore possible to apply the active compounds by the ultra-low-volume method or to inject the active compound formulation or the compound itself into the soil. The seed of the plants can also be treated.

When appling the active compounds of the invention as fungicides, the application rates can be varied within a substantial range, depending on the type of application. For the treatment of parts of plants, the application rates of active compound are, in general, between 0.1 and 10,000 g/ha, preferably between 10 and 1,000 g/ha. For the treatment of seed, the application rates of active compound are generally between 0.001 and 50 g per kilogram of seed, preferably between 0.01 and 10 g per kilogram of seed. For the treatment of the soil, the application rates of active compound are generally between 0.1 and 10,000 g/ha, preferably between 1 and 5,000 g/ha.

As already mentioned above, all plants and their parts may be treated in accordance with the invention. In a preferred embodiment, plant species and plant varieties which occur in the wild or which have been obtained by conventional biological breeding methods, such as hybridization or protoplast fusion, and parts of these plant species and plant varieties are treated. In another preferred embodiment, transgenic plants and plant varieties which have been obtained by genetic engineering methods, if appropriate in combination with conventional methods (genetically modified organisms) and parts of these plants and plant varieties are treated. The term “parts” and “parts of plants” or “plant parts” has been illustrated above.

Plants of the plant varieties which are commercially available or in use are especially preferably treated.

Depending on the plant species or plant varieties, their location and growth conditions (soils, climate, vegetation period, nutrition), superadditive (“synergistic”) effects may also occur as a result of the treatment in accordance with the invention. Thus, for example, reduced application rates and/or a widened spectrum of action and/or an increased action of the substances and compositions which can be used in accordance with the invention, better plant growth, increased tolerance to high or low temperatures, increased tolerance to drought or to water or soil salinity, increased flowering performance, facilitated harvesting, accelerated maturation, higher yields, higher quality and/or higher nutritional value of the harvested products, better storability and/or processability of the harvested products are possible, which exceed the actual effects to be expected.

The preferred transgenic plants or plant varieties (obtained by genetic engineering) to be treated in accordance with the invention include all plants which, owing to modification by recombinant means, contain genetic material which imparts particular advantageous valuable traits to these plants. Examples of such properties are better plant growth, increased tolerance to high or low temperatures, increased tolerance to drought or to water or soil salinity, increased flowering performance, facilitated harvesting, accelerated maturation, higher yields, higher quality and/or higher nutritional value of the harvested products, better storability and/or processability of the harvested products. Other, especially emphasized examples of such traits are an increased defence of the plants against animal and microbial pests such as against insects, mites, phytopathogenic fungi, bacteria and/or viruses, and an increased tolerance of the plants to certain herbicidal active compounds. Examples of transgenic plants which may be mentioned are important crop plants such as cereals (wheat, rice), maize, soya, potatoes, cotton, oilseed rape and fruit species (with the fruits apples, pears, citrus fruit and grapes), with particular emphasis being placed on maize, soya, potatoes, cotton and oilseed rape. Traits which are particularly emphasized are the increased defence of the plants against insects by toxins formed in the plants, in particular those which are generated by genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations) in the plants (hereinbelow “Bt plants”). Other traits which are particularly emphasized are the increased tolerance of the plants to certain herbicidal active compounds, for example imidazolinones, sulphonylureas, glyphosate or phosphinothricin (for example “PAT” gene). The genes which impart each of the desired traits may also occur in combination with each other in the transgenic plants. Examples of “Bt plants” which may be mentioned are varieties of maize, cotton, soya and potatoes, which are commercially available under the trade names YIELD GARD® (for example maize, cotton, soya), KnockOut® (for example maize), StarLink® (for example maize), Bollgard® (cotton), Nucoton® (cotton) and NewLeaf® (potatoes). Examples of herbicide-tolerant plants which may be mentioned are varieties of maize, cotton and soya which are commercially available under the trade names Roundup Ready® (tolerance to glyphosate, for example maize, cotton, soya), Liberty Link® (tolerance to phosphinothricin, for example oilseed rape), IMI® (tolerance to imidazolinones) and STS® (tolerance to sulphonylureas, for example maize). Herbicide-resistant plants (plants bred conventionally for herbicide tolerance) which may also be mentioned are the varieties which are commercially available under the name Clearfield® (for example maize). Naturally, these statements also apply to plant varieties which will be developed in the future or which will be marketed in the future and which have these genetic traits, or genetic traits to be developed in the future.

The plants stated can be treated particularly advantageously in accordance with the invention with the compounds of the general formula (I) or the active compound mixtures according to the invention. The preferred ranges stated above for the active compounds or mixtures also apply to the treatment of these plants. The treatment of plants with the compounds or mixtures specifically stated in the present text may be particularly emphasized.

The compositions used for the protection of industrial materials comprise the active compounds in general in an amount of 1 to 95%, preferably 10 to 75%.

The use concentrations of the active compounds according to the invention depend on the species and the distribution of the microorganisms to be controlled and on the composition of the material to be protected. The optimum use rate can be determined by test series. In general, the use concentrations are in the range of 0.001 to 5% by weight, preferably 0.05 to 1.0% by weight, based on the material to be protected.

The efficacy and the spectrum of action of the active compounds to be used according to the invention in the protection of materials, or of the compositions, concentrates or, quite generally, formulations which can be prepared from them, can be increased by adding, if appropriate, further antimicrobially active compounds, fungicides, bactericides, herbicides, insecticides or other active compounds to widen the spectrum of action or to achieve specific effects, such as, for example, additional protection against insects. These mixtures may have a wider spectrum of action than the compounds according to the invention.

When used against animal pests, the substances according to the invention in commercially available formulations and in the use forms which are prepared from these formulations may, again, be present as a mixture with synergists. Synergists are compounds by which the action of the active compounds is increased without it being necessary for the synergist added to be active itself.

The active compound content of the use forms prepared from the commercially available formulations can vary within wide ranges. The active compound concentration of the use forms can amount to from 0.0000001 to 95% by weight of active compound, preferably between 0.0001 and 1% by weight.

They are applied in a customary manner adapted to suit the use forms.

When applied against hygiene and stored-product pests, the active compound is distinguished by outstanding residual action on wood and clay and by good stability to alkali on limed substrates.

The invention is illustrated by the following examples.

### PREPARATION EXAMPLES

### Example 1

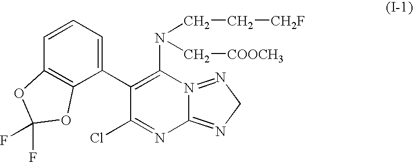

A mixture of 0.1 g (0.29 mmol) of 5,7-dichloro-6-(2,2-difluoro-1,3-benzodioxol-4-yl)-[1,2,4]-triazolo[1,5a]-pyrimidine and 0.04 g of (0.29 mmol) of (3-fluoropropyl)(methoxycarbonyl-methyl)-amine in 6.6 g of acetonitrile is admixed with 0.04 g (0.29 mmol) of potassium carbonate and stirred at room temperature for 16 hours. The reaction mixture is then filtered and concentrated under reduced pressure. The residue that remains is chromatographed on silica gel using hexane, ethyl acetate=3:1 as mobile phase. Concentration of the eluate gives 0.1 g (64.8% of theory) of the substance of the formula given above having a log P (S) of 2.93.

The triazolopyrimidines of the formula

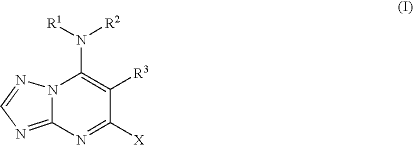

listed in Table 1 below are obtained in a similar manner.

TABLE 1

| Ex. No. | R¹ | R² | R³ | X | logP |
|---|---|---|---|---|---|
| 2 | —CH₂—CO—N(CH₃)₂ | 3-fluoropropyl | 2-chloro-5-trifluoro- | —Cl | 2.77 |
| | | | methylphenyl | | |
| 3 | —CH₂—CO—N(CH₃)₂ | 3-fluoropropyl | 2,5-bis-(trifluoro- | —Cl | 2.89 |
| | | | methyl)-phenyl | | |
| 4 | —CH₂—CO—N(CH₃)₂ | 3-fluoropropyl | 2,6-dichloro-3-fluoro- | —Cl | 3.01 |
| | | | 5-trifluoro- | | |
| | | | methylphenyl | | |
| 5 | —CH₂—CO—N(CH₃)₂ | 3-fluoropropyl | 2-fluoro-4-trifluoro- | —Cl | 2.71 |
| | | | methylphenyl | | |
| 6 | —CH₂—CO—N(CH₃)₂ | 3-fluoropropyl | 2-chloro-5-trifluoro- | —Cl | 3.14 |
| | | | methylthio-phenyl | | |
| 7 | —CH₂—CO—N(CH₃)₂ | 3-fluoropropyl | 2-chloro-3-trifluoro- | —Cl | 2.71 |
| | | | methylphenyl | | |
| 8 | —CH₂—CO—N(CH₃)₂ | 3-fluoropropyl | 2,2-difluoro-1,3- | —Cl | 2.52 |
| | | | benzodioxol-4-yl | | |
| 9 | —CH₂—CO—N(CH₃)₂ | 3-fluoropropyl | 2,3-difluoro-4- | —Cl | 2.86 |
| | | | trifluoromethyl-phenyl | | |
| 10 | —CH₂—COOCH₃ | 3-fluoropropyl | 2-chloro-5-trifluoro- | —Cl | 3.23 |
| | | | methylphenyl | | |
| 11 | —CH₂—COOCH₃ | 3-fluoropropyl | 2,5-bis-(trifluoro- | —Cl | |
| | | | methyl)-phenyl | | |
| 12 | —CH₂—COOCH₃ | 3-fluoropropyl | 2,6-dichloro-3-fluoro- | —Cl | 3.58 |
| | | | 5- | | |
| | | | trifluoromethylphenyl | | |
| 13 | —CH₂—COOCH₃ | 3-fluoropropyl | 2-fluoro-4-trifluoro- | —Cl | 3.17 |
| | | | methylphenyl | | |
| 14 | —CH₂—COOCH₃ | 3-fluoropropyl | 2-chloro-5-trifluoro- | —Cl | 3.57 |
| | | | methylthio-phenyl | | |
| 15 | —CH₂—COOCH₃ | 3-fluoropropyl | 2-chloro-3-trifluoro- | —Cl | 3.17 |
| | | | methylphenyl | | |
| 16 | —CH₂—COOCH₃ | 3-fluoropropyl | 2,3-difluoro-4- | —Cl | 3.29 |
| | | | trifluoromethyl-phenyl | | |
| 17 | —CH₂—CH(OCH₃)₂ | 3-fluoropropyl | 2-chloro-5-trifluoro- | —Cl | 3.54 |
| | | | methylphenyl | | |
| 18 | —CH₂—CH(OCH₃)₂ | 3-fluoropropyl | 2,5-bis-(trifluoro- | —Cl | |
| | | | methyl)-phenyl | | |
| 19 | —CH₂—CH(OCH₃)₂ | 3-fluoropropyl | 2,6-dichloro-3-fluoro- | —Cl | 3.82 |
| | | | 5-trifluoromethyl- | | |
| | | | phenyl | | |
| 20 | —CH₂—CH(OCH₃)₂ | 3-fluoropropyl | 2-fluoro-4-trifluoro- | —Cl | 3.41 |
| | | | methylphenyl | | |
| 21 | —CH₂—CH(OCH₃)₂ | 3-fluoropropyl | 2-chloro-5-trifluoro- | —Cl | 3.90 |
| | | | methylthio-phenyl | | |
| 22 | —CH₂—CH(OCH₃)₂ | 3-fluoropropyl | 2-chloro-3-trifluoro- | —Cl | 3.42 |
| | | | methylphenyl | | |
| 23 | —CH₂—CH(OCH₃)₂ | 3-fluoropropyl | 2,2-difluoro-1,3- | —Cl | 3.24 |
| | | | benzodioxol-4-yl | | |
| 24 | —CH₂—CH(OCH₃)₂ | 3-fluoropropyl | 2,3-difluoro-4- | —Cl | 3.55 |
| | | | trifluoromethyl-phenyl | | |
| 25 | 2,2-diethoxy-ethyl | 3-fluoropropyl | 2-chloro-5-trifluoro- | —Cl | 4.10 |
| | | | methylphenyl | | |
| 26 | 2,2-diethoxy-ethyl | 3-fluoropropyl | 2,5-bis-(trifluoro- | —Cl | |
| | | | methyl)-phenyl | | |
| 27 | 2,2-diethoxy-ethyl | 3-fluoropropyl | 2,6-dichloro-3-fluoro- | —Cl | 4.45 |
| | | | 5-trifluoro- | | |
| | | | methylphenyl | | |
| 28 | 2,2-diethoxy-ethyl | 3-fluoropropyl | 2-fluoro-4-trifluoro- | —Cl | 4.04 |
| | | | methylphenyl | | |
| 29 | 2,2-diethoxy-ethyl | 3-fluoropropyl | 2-chloro-5-trifluoro- | —Cl | 4.47 |
| | | | methylthio-phenyl | | |
| 30 | 2,2-diethoxy-ethyl | 3-fluoropropyl | 2-chloro-3-trifluoro- | —Cl | 4.00 |
| | | | methylphenyl | | |
| 31 | 2,2-diethoxy-ethyl | 3-fluoropropyl | 2,2-difluoro-1,3- | —Cl | 3.81 |
| | | | benzodioxol-4-yl | | |
| 32 | 2,2-diethoxy-ethyl | 3-fluoropropyl | 2,3-difluoro-4- | —Cl | 4.16 |
| | | | trifluoromethyl-phenyl | | |
| 33 | —CH₂COCH₃ | 3-fluoropropyl | 2-chloro-5-trifluoro- | —Cl | 3.07 |
| | | | methylphenyl | | |
| 34 | 2,2-dimethoxypropyl | 3-fluoropropyl | 2,5-bis-(trifluoro- | —Cl | |
| | | | methyl)-phenyl | | |
| 35 | 2,2-dimethoxypropyl | 3-fluoropropyl | 2,6-dichloro-3-fluoro- | —Cl | |
| | | | 5-trifluoromethyl- | | |
| | | | phenyl | | |
| 36 | —CH₂COCH₃ | 3-fluoropropyl | 2-fluoro-4-trifluoro- | —Cl | 3.01 |
| | | | methylphenyl | | |
| 37 | —CH₂COCH₃ | 3-fluoropropyl | 2-chloro-5-trifluoro- | —Cl | 3.42 |
| | | | methylthio-phenyl | | |
| 38 | —CH₂COCH₃ | 3-fluoropropyl | 2-chloro-3-trifluoro- | —Cl | 3.01 |
| | | | methylphenyl | | |
| 39 | —CH₂COCH₃ | 3-fluoropropyl | 2,2-difluoro-1,3- | —Cl | 2.80 |
| | | | benzodioxol-4-yl | | |
| 40 | —CH₂COCH₃ | 3-fluoropropyl | 2,3-difluoro-4- | —Cl | 3.14 |
| | | | trifluoromethyl-phenyl | | |
| 41 | 2-bromoallyl | 3-fluoropropyl | 2-chloro-5- | —Cl | 3.92 |
| | | | trifluoromethylphenyl | | |
| 42 | 2-bromoallyl | 3-fluoropropyl | 2,5-bis-(trifluoro- | —F | 3.84 |
| | | | methyl)-phenyl | | |
| 43 | 2-bromoallyl | 3-fluoropropyl | 2,6-dichloro-3-fluoro- | —Cl | 4.27 |
| | | | 5-trifluoromethyl- | | |
| | | | phenyl | | |
| 44 | 2-bromoallyl | 3-fluoropropyl | 2-fluoro-4-trifluoro- | —Cl | 3.85 |
| | | | methylphenyl | | |
| 45 | 2-bromoallyl | 3-fluoropropyl | 2-chloro-5-trifluoro- | —Cl | 4.36 |
| | | | methylthiophenyl | | |
| 46 | 2-bromoallyl | 3-fluoropropyl | 2-chloro-3-trifluoro- | —F | 3.73 |
| | | | methylphenyl | | |
| 47 | 2-bromoallyl | 3-fluoropropyl | 2,2-difluoro-1,3- | —Cl | 3.69 |
| | | | benzodioxol-4-yl | | |
| 48 | 2-bromoallyl | 3-fluoropropyl | 2,3-difluoro-4- | —Cl | 3.94 |
| | | | trifluoromethylphenyl | | |
| 49 | 2-chloroallyl | 3-fluoropropyl | 2-chloro-5-trifluoro- | —Cl | 3.84 |
| | | | methylphenyl | | |
| 50 | 2-chloroallyl | 3-fluoropropyl | 2,5-bis-(trifluoro- | —F | 3.77 |
| | | | methyl)-phenyl | | |
| 51 | 2-chloroallyl | 3-fluoropropyl | 2,6-dichloro-3-fluoro- | —Cl | 4.19 |
| | | | 5-trifluoromethyl- | | |
| | | | phenyl | | |
| 52 | 2-chloroallyl | 3-fluoropropyl | 2-fluoro-4-trifluoro- | —Cl | 3.77 |
| | | | methylphenyl | | |
| 53 | 2-chloroallyl | 3-fluoropropyl | 2-chloro-5-trifluoro- | —Cl | 4.27 |
| | | | methylthio-phenyl | | |
| 54 | 2-chloroallyl | 3-fluoropropyl | 2-chloro-3-trifluoro- | —F | 3.66 |
| | | | methylphenyl | | |
| 55 | 2-chloroallyl | 3-fluoropropyl | 2,2-difluoro-1,3- | —Cl | 3.61 |
| | | | benzodioxol-4-yl | | |
| 56 | 2-chloroallyl | 3-fluoropropyl | 2,3-difluoro-4- | —Cl | 3.86 |
| | | | trifluoromethylphenyl | | |
| 57 | —CH₂—CF₃ | 3-fluoropropyl | 2-chloro-5-trifluoro- | —Cl | |
| | | | methylphenyl | | |
| 58 | —CH₂—CF₃ | 3-fluoropropyl | 2,5-bis-(trifluoro- | —Cl | |
| | | | methyl)-phenyl | | |
| 59 | —CH₂—CF₃ | 3-fluoropropyl | 2,6-dichloro-3-fluoro- | —Cl | |
| | | | 5-trifluoromethyl- | | |
| | | | phenyl | | |
| 60 | —CH₂—CF₃ | 3-fluoropropyl | 2-fluoro-4-trifluoro- | —Cl | |
| | | | methylphenyl | | |
| 61 | —CH₂—CF₃ | 3-fluoropropyl | 2-chloro-5-trifluoro- | —Cl | |
| | | | methylthio-phenyl | | |
| 62 | —CH₂—CF₃ | 3-fluoropropyl | 2-chloro-3-trifluoro- | —Cl | |
| | | | methylphenyl | | |
| 63 | —CH₂—CF₃ | 3-fluoropropyl | 2,2-difluoro-1,3- | —Cl | |
| | | | benzodioxol-4-yl | | |
| 64 | —CH₂—CF₃ | 3-fluoropropyl | 2,3-difluoro-4- | —Cl | |
| | | | trifluoromethylphenyl | | |
| 65 | 2,2,2-trifluoro-1- | 3-fluoropropyl | 2-chloro-5-trifluoro- | —Cl | |
| | methylethyl | | methylphenyl | | |
| 66 | 2,2,2-trifluoro-1- | 3-fluoropropyl | 2,5-bis-(trifluoro- | —Cl | |
| | methylethyl | | methyl)-phenyl | | |
| 67 | 2,2,2-trifluoro-1- | 3-fluoropropyl | 2,6-dichloro-3-fluoro- | —Cl | |
| | methylethyl | | 5-trifluoromethyl- | | |
| | | | phenyl | | |
| 68 | 2,2,2-trifluoro-1- | 3-fluoropropyl | 2-fluoro-4-trifluoro- | —Cl | |
| | methylethyl | | methylphenyl | | |
| 69 | 2,2,2-trifluoro-1- | 3-fluoropropyl | 2-chloro-5-trifluoro- | —Cl | |
| | methylethyl | | methylthio-phenyl | | |
| 70 | 2,2,2-trifluoro-1- | 3-fluoropropyl | 2-chloro-3-trifluoro- | —Cl | |
| | methylethyl | | methylphenyl | | |
| 71 | 2,2,2-trifluoro-1- | 3-fluoropropyl | 2,2-difluoro-1,3- | —Cl | |
| | methylethyl | | benzodioxol-4-yl | | |
| 72 | 2,2,2-trifluoro-1- | 3-fluoropropyl | 2,3-difluoro-4- | —Cl | |
| | methylethyl | | trifluoromethylphenyl | | |
| 73 | —CH₂—CH(OCH₃)₂ | 3-fluoropropyl | 2,4-difluoro-6- | —Cl | 3.22 |
| | | | trifluoromethyl-phenyl | | |
| 74 | —CH₂—COOCH₃ | 3-fluoropropyl | 2,4-difluoro-6- | —Cl | 2.92 |
| | | | trifluoromethylphenyl | | |

### Preparation of Starting Materials

### Example 75

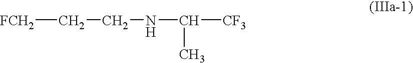

At room temperature, a mixture of 10.0 g (88.43 mmol) of 2,2,2-trifluoro-1-methyl-ethyl-amine and 6.11 g (44.22 mmol) of potassium carbonate in 78.00 g of acetonitrile is, with stirring, admixed with 12.47 g (88.43 mmol) of 1-fluoro-3-bromo-propane over a period of 30 minutes. After the addition has ended, the reaction mixture is stirred at room temperature for 16 hours and then filtered. The filtrate is initially concentrated under reduced pressure and then distilled under atmospheric pressure. In this manner, 2.1 g (12.34% of theory) of (3-fluoropropyl)-(2,2,2-trifluoro-1-methyl-ethyl)-amine are obtained in the form of a liquid.

### Example 76

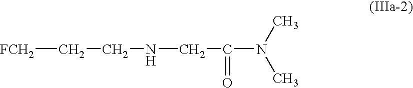

A mixture of 10.0 g (40.11 mmol) of 3-fluoro-propyl-ammonium tosylate and 156.0 g of acetonitrile is admixed with 8.32 g (60.17 mmol) of potassium carbonate and stirred at room temperature for 1 hour. 4.88 g (40.11 mmol) of 2-chloro-N,N-dimethylacetamide are then added, and the mixture is stirred at room temperature for 24 hours. The reaction mixture is then filtered. The filtratre is concentrated under reduced pressure. The residue that remains is distilled under greatly reduced pressure. In this manner, 1.3 g (17.98% of theory) of (3-fluoropropyl)-(N,N-dimethylcarbamoyl-methyl)-amine are obtained in the form of a liquid.

The amines of the formula

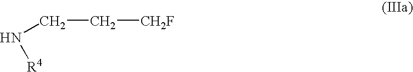

listed in Table 2 below are likewise obtained by the methods given above.

TABLE 2

| Ex. No. | R⁴ | Boiling point (° C.) |
|---|---|---|
| 77 | 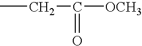 | 97-103/19 mbar |
| 78 | —CH₂—CF₃ | 90-103 |
| | | 70/15 mbar |
| 79 | 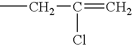 | 70/15 mbar |
| 80 | 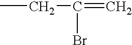 | 90/16 mbar |
| 81 | 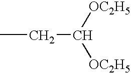 | 62-66/1.3 mbar |
| 82 | 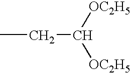 | 52-55/2 mbar |
| 83 | 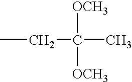 | 48-49/2 mbar |

### Example 84 FCH₂—CH₂—CH₂—OH

A mixture of 235.5 g (4.0 mol) of potassium fluoride and 787.5 g of triethylene glycol is stirred at 100° C. for 1 hour. 200 ml of triethylene glycol are then distilled off, the mixture is allowed to cool to room temperature and 128.0 g (1.353 mol) of 3-chloro-1-propanol are added. The reaction mixture is heated at 180° C. for 2 hours and then distilled under reduced pressure. This gives 67.6 g (63.95% of theory) of 3-fluoro-1-propanol in the form of a liquid.

### Example 85 FCH₂—CH₂—CH₂—Br (IV)

Over a period of two hours, a mixture of 67.6 g (0.865 mol) of 3-fluoro-1-propanol in 190 g of dimethylformamide is, at room temperature, with cooling and with stirring, mixed with 200 g (0.962 mol) of thionyl bromide. After the addition has ended, stirring at room temperature is continued for 2 hours, and the mixture is then distilled under reduced pressure. This gives 47.2 g (37.5% of theory) of 1-fluoro-3-bromo-propane in the form of a liquid of boiling point 100-103° C.

### Example 86

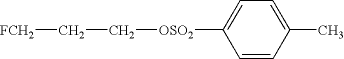

At 0° C., a mixture of 2.81 g (36 mmol) of 3-fluoro-1-propanol and 30 ml of pyridine is, with stirring, admixed with 13.73 g (72 mmol) of 4-tosyl chloride over a period of 5 minutes. After the addition has ended, the reaction mixture is stirred at 0° C. for another hour, 2.7 ml of water are then added at −10° C. over a period of 5 minutes and subsequently 30 ml of water are added. For work-up, the reaction mixture is poured into 1 litre of water and the resulting mixture is extracted three times with ether. The combined organic phases are washed twice with water and then concentrated under reduced pressure. This gives 7.1 g (84.9% of theory) of 3-fluoro-propyl tosylate.

### Beispiel 87

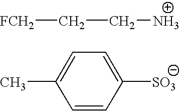

Over a period of 2.5 hours, 84.0 g (4.932 mol) of ammonia are introduced at room temperature into a solution of 96.0 g (415 mmol) of 3-fluoro-propyl tosylate in 500 ml of ethanol. The reaction mixture is then initially stirred at room temperature for 16 hours and then under reflux for another hour. For work-up, the reaction mixture is concentrated under reduced pressure. The residue that remains is washed successively with ethyl acetate and with ether. This gives 43.2 g (41.67% of theory) of 3-fluoro-propyl-ammonium tosylate.

1. Triazolopyrimidines of the formula 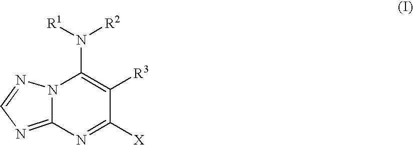
in which
R¹ and R² independently of one another represent alkyl which is mono- or polysubstituted by identical or different substituents from the group consisting of halogen, cyano, nitro, amino, hydroxyl, formyl, carboxyl, carbamoyl, thiocarbamoyl, alkoxy, alkylthio, alkylsulphinyl, alkylsulphonyl, alkenyloxy, alkinyloxy, halogenoalkoxy, halogenoalkylthio, halogenoalkylsulphinyl, halogenoalkylsulphonyl, halogenoalkenyloxy, halogenoalkenylthio, halogenoalkenylsulphinyl, halogenoalkenylsulphonyl, halogenoalkinyloxy, halogenoalkinylthio, halogenoalkinylsulphinyl, halogenoalkinylsulphonyl, alkylamino, dialkylamino, alkylcarbonyl, alkylaminocarbonyl, dialkylaminocarbonyl, alkylaminothiocarbonyl, dialkylaminothiocarbonyl, alkylcarbonyloxy, alkoxycarbonyl, hydroximinoalkyl, alkoximinoalkyl, cycloalkyl, aryl and heterocyclyl,
or
represent optionally substituted alkenyl, optionally substituted alkinyl or optionally substituted cycloalkyl,
R³ represents optionally substituted heterocyclyl or represents substituted aryl, where at least one substituent from the following group of radicals is present
formyl, alkinyl, halogenoalkyl, halogenoalkoxy, halogenoalkylthio, halogenoalkylsulphinyl, halogenoalkylsulphonyl, halogenoalkenyl, halogenoalkinyl, halogenoalkenyloxy, halogenoalkinyloxy, alkylamino, dialkylamino, alkylcarbonyl, alkoxycarbonyl, hydroximinoalkyl or alkoximinoalkyl,
doubly attached alkylene having 3 to 6 carbon atoms which may be mono- or polysubstituted by identical or different substituents from the group consisting of halogen, alkyl having from 1 to 4 carbon atoms and halogenoalkyl having 1 to 4 carbon atoms and 1 to 9 identical or different halogen atoms,
doubly attached oxyalkylene having 2 to 5 carbon atoms which may be mono- or polysubstituted by identical or different substituents from the group consisting of halogen, alkyl having from 1 to 4 carbon atoms and halogenoalkyl having 1 to 4 carbon atoms and 1 to 9 identical or different halogen atoms,
doubly attached dioxyalkylene having 1 to 4 carbon atoms, where the oxygen atoms are not adjacent and where the alkylene chain may be mono- or polysubstituted by identical or different substituents from the group consisting of halogen, alkyl having 1 to 4 carbon atoms and halogenoalkyl having 1 to 4 carbon atoms and 1 to 9 identical or different halogen atoms,
and
X represents halogen.

2. Triazolopyrimidines of the formula (I) according to claim 1 in which
R¹ represents methyl, ethyl, n-propyl, isopropyl, n-, i-, sec- or tert-butyl, where these radicals are mono- to trisubstituted by identical or different substituents from the group consisting of fluorine, chlorine, bromine, methoxy, ethoxy, acetyl, dimethylaminocarbonyl, methoxycarbonyl, ethoxycarbonyl or phenyl,
or
R¹ represents propenyl, butenyl, pentenyl or hexenyl, where these radicals may be mono- to trisubstituted by identical or different substituents from the group consisting of fluorine, chlorine and bromine,
R² represents methyl, ethyl, n-propyl, isopropyl, n-, i-, sec- or tert.-butyl, where these radicals are mono- to trisubstituted by identical or different substituents from the group consisting of fluorine, chlorine and bromine,
R³ represents phenyl which is mono- to tetrasubstituted by identical or different substituents from the group consisting of fluorine, chlorine, trifluoromethyl and trifluoromethylthio,
where at least one substituent is a trifluoromethyl or trifluoromethylthio group, or
R³ represents the radical of the formula 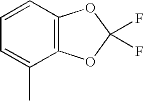
and
X represents fluorine or chlorine.

3. Process for preparing triazolopyrimidines of the formula (I) according to claim 1, characterized in that dihalogeno-triazolopyrimidines of the formula 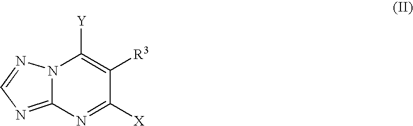
in which
R³ and X have the meanings given above and
Y represents halogen,
are reacted with amines of the formula 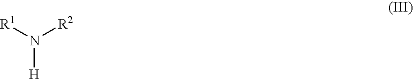
in which
R¹ and R² have the meanings given above,
if appropriate in the presence of a diluent and if appropriate in the presence of an acid acceptor.

4. Compositions for controlling undesirable microorganisms and animal pests, characterized in that they comprise at least one triazolo pyrimidine of the formula (I) according to claim 1, in addition to extenders and/or surfactants.

5. Use of triazolopyrimidines of the formula (I) according to claim 1 for controlling undesirable microorganisms and animal pests.

6. Method for controlling undesirable microorganisms and animal pests, characterized in that triazolopyrimidines of the formula (I) according to claim 1 are applied to the undesirable microorganisms or animal pests and/or their habitat.

7. Process for preparing compositions for controlling undesirable microorganisms and animal pests, characterized in that triazolopyrimidines of the formula (I) according to claim 1 are mixed with extenders and/or surfactants.

8. Amines of the formula 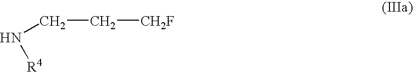
in which
R⁴ represents the radicals of the formulae

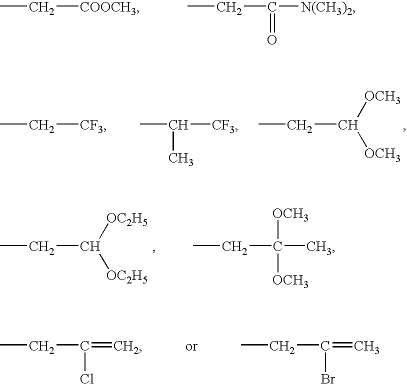

9. Process for preparing amines of the formula (IIIa) according to claim 8, characterized in that
either
a) 1-fluoro-3-bromo-propane of the formula
FCH₂—CH₂—CH₂—Br (IV)
is reacted with amines of the fomula
H₂N—R⁴ (V)
R⁴ has the meanings given above,
or with acid addition salts of amines of the formula (V)
in the presence of an inert organic diluent and in the presence of an acid binder,
or
b) 3-fluoro-propyl-amine of the formula,
FCH₂—CH₂—CH₂—NH₂ (VI),
if appropriate in the form of an acid addition salt,
is reacted with halogen compounds of the formula
R⁴—Hal (VII)
in which
R⁴ has the meanings given above and
Hal represents chlorine or bromine,
in the presence of an inert diluent and in the presence of an acid binder.
